(19) United States
(12) Plant Patent Application Publication
(10) Pub. No.: US 2018/0324992 P1
(43) Pub. Date: Nov. 8, 2018

(54) Strawberry plant named 'Alice'

(50) ;

(71) Applicant: SWEET DARLING SALES, INC., Aptos, CA (US)

(72) Inventor: John Larse, Watsonville, CA (US)

(21) Appl. No.: 15/932,838

(22) Filed: May 4, 2018

Related U.S. Application Data
(60) Provisional application No. 62/501,253, filed on May 4, 2017.

(51) Int. Cl. A01H 6/74

(52) U.S. Cl. CPC A01H 6/7409
USPC PLT/208

(57) ABSTRACT

The present invention provides new and distinct strawberry plant designated as ‘Alice’ (a.k.a. ‘109180’).

### CROSS-REFERENCE TO RELATED APPLICATION

The present application claims priority to, and the benefit of U.S. Provisional Patent Application No. 62/501,253, filed on May 4, 2017, which is herein incorporated by reference in its entirety.

Latin name of the genus and species: Fragaria x ananassa.

Varietal denomination: ‘Alice’ (a.k.a. ‘109180’).

### BACKGROUND OF THE INVENTION

The present invention relates to new and distinct strawberry plant designated as ‘Alice’ (a.k.a. ‘109180’).

‘Alice’ (a.k.a. ‘109180’) is the result of a controlled-cross between a female parent cultivar designated 105218 and a male parent cultivar designated 102850 made by the Inventor and was first fruited in Watsonville, Calif. growing fields. Following selection and during testing, the plant was originally designated ‘109180’ and subsequently named ‘Alice’. ‘Alice’ is a day-neutral plant.

This new strawberry plants was asexually reproduced via runners (stolons) by the inventor at Watsonville, Calif. Asexual propagules from the original source have been tested in Watsonville growing fields and to a limited extent, grower fields in high elevation. The properties of this plant were found to be transmissible by such asexual reproduction. The plant is stable and reproduces true to type in successive generations of asexual reproduction.

### BRIEF SUMMARY OF THE INVENTION

This invention relates to new and distinctive strawberry plant designated as ‘Alice’ (a.k.a. ‘109180’). The plant is primarily adapted to the climate and growing conditions of the central coast of California. This region provides the necessary temperatures required for it to produce a strong vigorous plant and to remain in fruit production from March through October. The nearby Pacific Ocean provides the needed humidity and moderate day temperatures and evening chilling to maintain fruit quality for the production months.

The following traits and photographs in combination distinguish strawberry plant ‘Alice’ from known strawberry plants. Plants for the botanical measurements in the present application were grown as annuals. Any color references are made to The Royal Horticultural Society Colour Chart, 1995 Edition, except where general terms of ordinary dictionary significance are used.

TABLE 1

| | Characteristic | Description |
|---|---|---|
| General | Plant Habit | Perennial |
| | Plant Growth Habit | Upright |
| | Day Length | Day Neutral |
| | Height (cm) | 34 |
| | Width (cm) | 35 |
| | Density of foliage | low |
| | Plant vigor | moderate |
| Leaf | Terminal leaflet width (mm) | 93 |
| | Terminal leaflet length (mm) | 86 |
| | No. teeth/terminal leaflet | 24 |
| | Shape of the terminal leaflet base | acute |
| | Shape of terminal leaflet in cross-section | concave |
| | Margin description of the terminal | serrate to crenate |
| | Color of upper side of leaves | 137A |
| | Color of lower side of leaves | 139C |
| | Leaf blistering | absent |
| | Leaf glossiness | medium |
| Limbs | Petiole length (cm) | 20 |
| | Petiole diameter (mm) | 4.8 |
| | Petiole color | 145A |
| | Petiolule length (cm) | 0.95 |
| | Petiolule diameter (mm) | 3.83 |
| | Stipule length (cm) | 4 |
| | Stipule width (mm) | 1.4 |
| | Stipule anthocyanin | present |
| | Stipule color (color code) | 145C |
| | Pedicel color (color code) | 145A |
| | Attitude of hairs on | upwards |
| | petiole and pedicel | |
| Inflorescence | Inflorescence position relative to foliage | above |
| | flower arrangement of petals | free |
| | Petal length (cm) | 1 |
| | Petal width (cm) | 1.1 |
| | Petal number per flower | 5 to 6 |
| | Upper Petal color | 155C |
| | Lower Petal color | 155D |
| | peduncle size | medium |
| | Calyx diameter (cm) | 3.7 |
| | Corolla diameter (cm) | 2.6 |
| | Sepal length (cm) | 1.3 |
| | Sepal width (cm) | 0.5 |
| | Time of flowering | March |
| | (50% of plants in bloom) | |
| | Shape of stigma | capitate |
| | Color of stigma | 172A |
| | Length of style (mm) | 1.12 |
| | COlor of style | 6A |
| | Color of the ovary | 145B |
| | Length of the stamens (mm) | 4.6 |
| | Number of stamen | 26 |
| | Shape of anther | dorsifixed |
| | Size of anther | medium |
| | Color of anther | 12A |
| | Amount of pollen | moderate to high |
| | COlor of pollen | 22A |
| | Color of filament | 149D |
| | Length of filament (mmm) | 2.73 |
| | Number of flower per truss | 4 to 6 |
| Stolon | Stolon n umber | 3 to 11 |
| | Stolon anthocyanin | 181A |
| | Widest diameter of stolon | 3.3 |
| | At leaf attachment (mm) | |
| | Stolon color | 145A |
| Fruit | Number of fruit per truss | 3 to 5 |
| | Fruit length (cm) | 4.7 |
| | Fruit width (cm) | 4.1 |
| | Fruit skin color | 45A |
| | Fruit fresh color excluding core | 41A |
| | Fruit core color | 49A |
| | Fruit weight (g) | 28 |
| | Predominant fruit shape | long conic to long |
| | | wedge |
| | Shape difference between primary & | No shape |
| | secondary fruits | difference |
| | Width of band without of achenes | medium |
| | Fruit glossiness | medium |
| | Position of achenes | even |
| | Achene color | 149A |
| | Achenes per fruit | 308 |
| | Achene weight (g) | 0.22 |
| | Position of calyx | inserted |
| | Level of adherence of calyx | strong |
| | Color of calyx | 137C |
| | Firmness of flesh | firm |
| | Evenness of flesh color | nearly even |
| | Sweetness | 8 |
| | pH | 3.46 |
| | Yield (grams per plant per season) | 2206 |

‘Alice’ is distinguished from its paternal parent by its larger fruit size, greater ease of harvest, and its presentation of shorter and thicker petioles. ‘Alice’ is distinguished from its maternal parent by its larger fruit size, greater yield volume, and longer flower stems. ‘Alice’ is similar to the UC Davis variety ‘Monterey’ (U.S. Plant Pat. No. 19,767), but possesses several distinguishing traits from Monterey. ‘Alice’ is distinguished from ‘Monterey’ by its greater ease of harvest, smaller plant size, and higher berry yield per plant. ‘Alice’ presents shorter and thicker petioles than ‘Monterey’. ‘Alice’ has a lower cull rate than ‘Monterey’ and both of its parents. ‘Albion’ (U.S. Plant Pat. No. 16,228) is a commercial strawberry variety that is similar to, but distinguished from ‘Alice’. The fruit yield of ‘Alice’ exceeds the yield of ‘Albion’ in multiple annual testing cycles performed at the breeders test location in Watsonville Calif.

In addition, ‘Alice’ scored better than it's parents for percent marketable fruit of the sum of all fruit produced in Watsonville Calif. test plots. Compared to its parents, ‘Alice’ was observed to withstand adverse post harvest ambient temperatures over several days, demonstrating a putative longer shelf life as well as less susceptibility to the discoloration effect associated with bruising that occurs during the hand harvesting procedure. The fruit of each of the parents of the strawberry plant variety named ‘Alice’ was observed to be lighter in redness coloration “Alice”. One parent of ‘Alice’, a strawberry plant variety named “Victor”, was determined to be a short day variety, whereas ‘Alice’ has been determined to be a day neutral type when grown in the breeder's Watsonville Calif. test farm. The color of the foliage of ‘Victor’ is a lighter green than it's offspring ‘Alice’. ‘Victor’ produces more fruit over the fruiting season than it's offspring ‘Alice’, however ‘Alice’ produces fruit during a longer period of time than it's short-day parent ‘Victor’. The other parent of ‘Alice’, a strawberry plant variety named ‘Zaralina’ produces much less fruit than ‘Alice’, and the plant structure of ‘Zaralina’ is smaller and more compact than ‘Alice’. The strawberry plant variety named ‘Alice’ produces more stolon in the fruiting field than either of it's parents when grown in the breeder's Watsonville location.

### DESCRIPTION OF THE DRAWINGS

The accompanying color photographs depict various characteristics of the cultivars as nearly true as possible to make color reproductions.

FIG. 1 shows ripe and near-ripe fruits of ‘Alice’ about 6-month old.

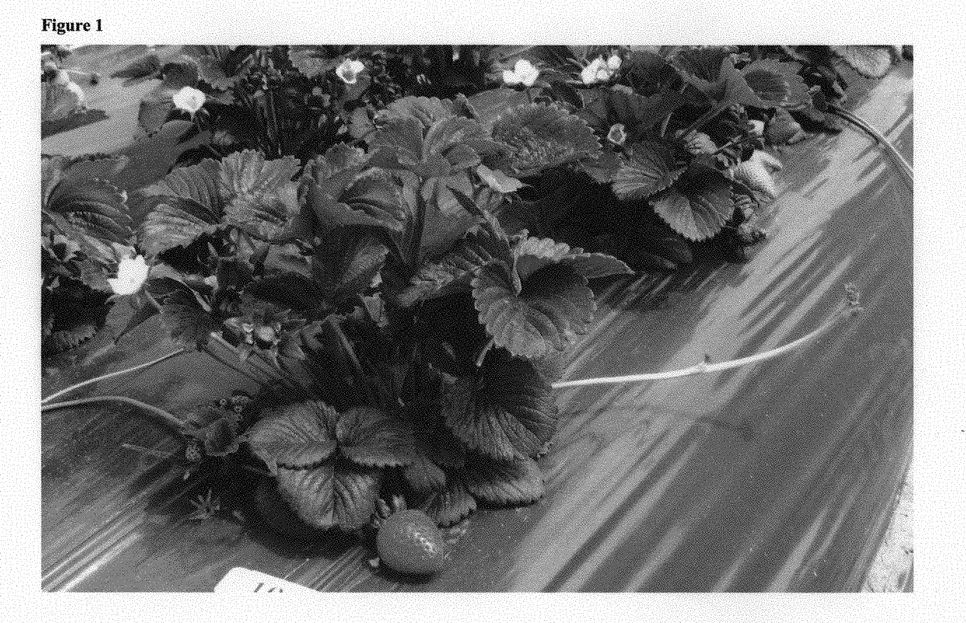

FIG. 2 shows ‘Alice’ plants growing in the field with multiple fruits.

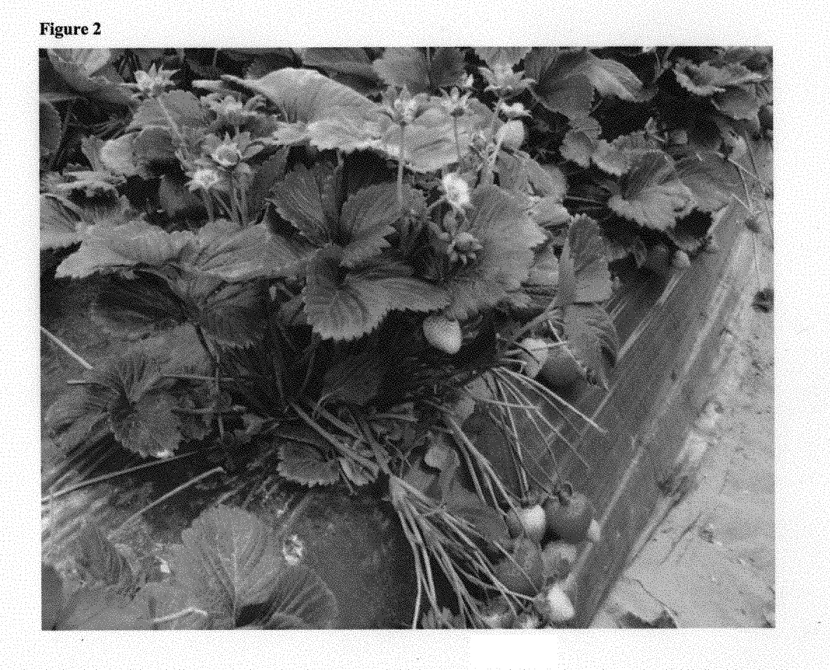

FIG. 3 shows the front side of a leaf of ‘Alice’ plants.

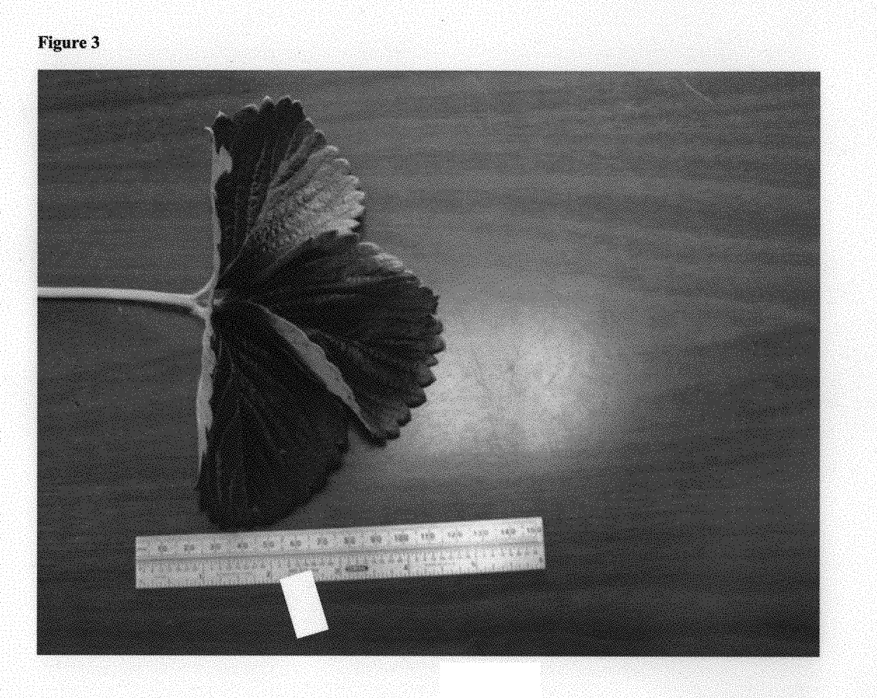

FIG. 4 shows the back side of a leaf of ‘Alice’ plants.

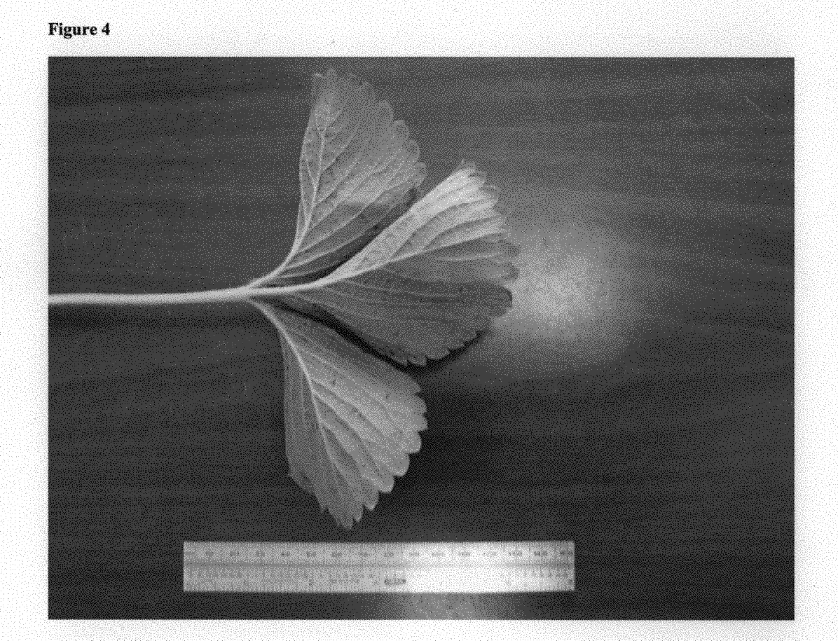

FIG. 5 shows fruits of ‘Alice’ plants.

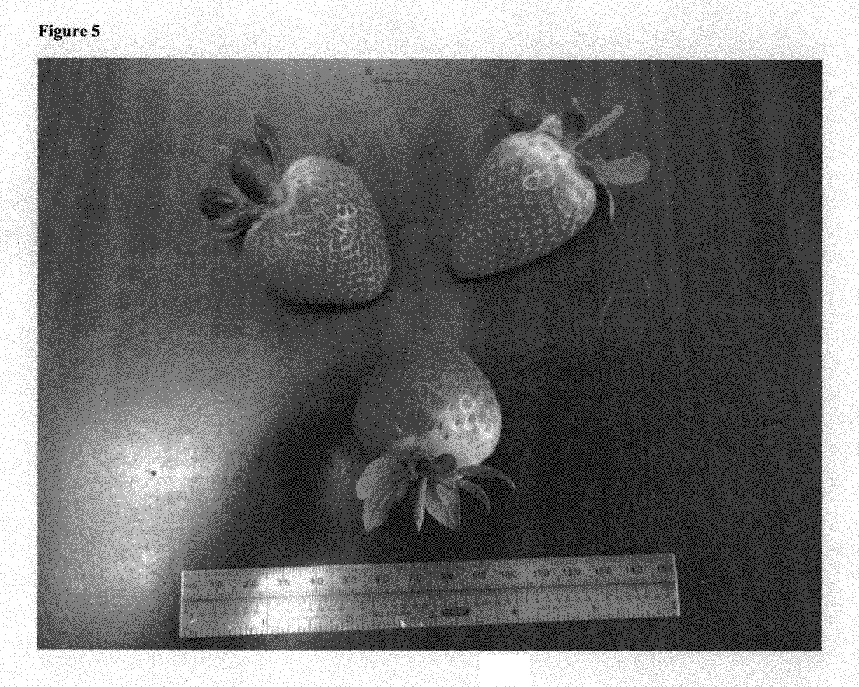

FIG. 6 shows fruits of ‘Alice’ plants cut in half.

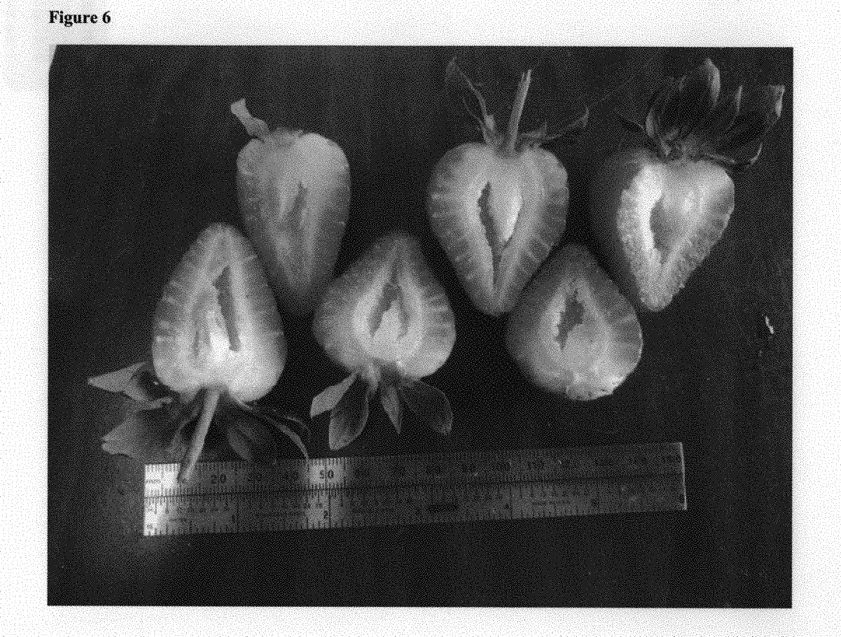

1. A new and distinct cultivar of strawberry plant named ‘Alice’, substantially as shown and described herein.
